(19) United States
(12) Patent Application Publication
(10) Pub. No.: US 2024/0325005 A1
(43) Pub. Date: Oct. 3, 2024

(54) DOUBLE ENDOSCOPIC LOOP FOR LAPAROSCOPIC SURGERY

(71) Applicant: KING FAISAL UNIVERSITY, Al-Ahsa (SA)

(72) Inventor: Abdulrahman Saleh ALMULHIM, Al-Ahsa (SA)

(21) Appl. No.: 18/191,431

(22) Filed: Mar. 28, 2023

(51) Int. Cl. A61B 17/00

(52) U.S. Cl. CPC A61B 17/00234; A61B 2017/00296; A61B 2017/00823

(57) ABSTRACT

A device having endoscopic loops for laparoscopic surgery, such as an appendectomy, and methods of using the device. The device includes a longitudinal body having a proximal end and a distal end, and a first loop spaced apart from a second loop at the distal end. The first loop is configured to secure an appendage at a first point and the second loop secures the appendage at a second point. A handle is located at the proximal end and is connected to the first loop and the second loop such that when the handle is pulled away from the longitudinal body the first loop further secures the appendage at the first point and the second loop further secures the appendage at the second point.

### BACKGROUND

### 1. Field

The present disclosure relates to laparoscopic surgery, and particularly to double endoscopic loop laparoscopic surgery.

### 2. Description of the Related Art

Laparoscopic surgery is a surgical procedure that allows access to the inside of a patient's body without making a large incision. This type of surgery is sometimes coined as keyhole surgery since it only requires a small incision and is minimally invasive compared to other types of surgeries. Small incisions are made in the patient to allow access into the body. A tube is placed through the incision and into the body. Gas is pumped into the body through the tube. A laparoscope, that includes a small camera and light, is inserted into the body, allowing for images to be displayed on a monitor. Small surgical tools can then be inserted into the body through other small incisions allowing for medical procedures to be performed.

It can be challenging to perform laparoscopic surgery due to the small size of the incisions. The surgical tools must be small and it can be difficult to operate the tools in such a confined space. The patient may also prefer not to have so many incisions.

An appendectomy is a common emergency surgery requiring the removal of the appendix, which is a small thin pouch attached to the large intestine. The appendix is located in the lower right part of the belly. Removal of the appendix can become necessary when the appendix becomes sore, swollen and/or infected.

Performing laparoscopic surgery to remove the appendix will require incisions in the lower right part of the belly to pump gas into the body and insert a laparoscope. Surgical tools that tie off the appendix from the large intestine and to remove the appendix will then need to be inserted into the body. Two endoscopic loops are sometimes used to secure the base of the appendix before cutting. The surgery can be difficult because of the number of surgical instruments that need to be used. It will take up a considerable amount of space and will require multiple incisions. The number of surgical instruments will also be hard to handle.

Thus, a device to assist with laparoscopic surgery solving the aforementioned problems is desired.

### SUMMARY

A device having a double endoscopic loop for laparoscopic surgery will reduce the number of surgical instruments needed in surgeries such as an appendectomy and will also simplify the procedure.

Accordingly, a device having endoscopic loops for laparoscopic surgery, in one embodiment, includes a longitudinal body having a proximal end and a distal end. A first loop can be spaced apart from a second loop at the distal end. The first loop can be configured, in use, for securing an appendage at a first point, and the second loop can be configured, in use, for securing the appendage at a second point. A handle can be located at the proximal end. The handle can be connected to the first loop and the second loop such that when the handle is pulled away from the longitudinal body the first loop further secures the appendage at the first point and the second loop further secures the appendage at the second point.

The first point can be located at the base of the appendage and the second point can be located at a distance on the appendage spaced apart from the first point.

The first loop can be spaced apart from the second loop at a distance of about 4 cm.

The longitudinal body in some embodiments can be about 30 cm long.

The handle can include a first looped handle located on the left side of the longitudinal body and a second looped handle located on the right side of the longitudinal body.

The first looped handle, in one embodiment, can extend about 5 cm to the left of the longitudinal body and the second looped handle can extend about 5 cm to the right of the longitudinal body.

The device can further include wires that extend from the handle to the first loop and the second loop such that when the handle is pulled away from the longitudinal body the first loop further secures the appendage at the first point and the second loop further secures the appendage at the second point.

In another embodiment, the present subject matter relates to a method of assisting in an appendectomy in a patient, the method comprising: making an incision in a body of the patient above the patient's appendix; inserting a device having endoscopic loops through the incision into the body of the patient, the device comprising: a longitudinal body having a proximal end and a distal end, a first loop spaced apart from a second loop at the distal end, and a handle located at the proximal end, the handle connected to the first loop and the second loop; securing the appendix at a first point using the first loop and securing the appendix at a second point using the second loop; and pulling the handle away from the longitudinal body such that the first loop further secures the appendix at the first point and the second loop further secures the appendix at the second point.

These and other features of the present subject matter will become readily apparent upon further review of the following specification.

### BRIEF DESCRIPTION OF THE DRAWINGS

FIG. 1 is an illustration of a double endoscopic loop for laparoscopic surgery.

FIG. 2 is an illustration of the double endoscopic loop inside of a patient.

Similar reference characters denote corresponding features consistently throughout the attached drawings.

### DETAILED DESCRIPTION OF THE PREFERRED EMBODIMENTS

A double endoscopic loop for laparoscopic surgery incudes two loops that can be inserted at one time. An appendectomy, in some instances, makes use of two endoscopic loops to secure the base of the appendix before cutting. A device that inserts two loops at once will significantly simplify the surgery by reducing the number of surgical tools needed.

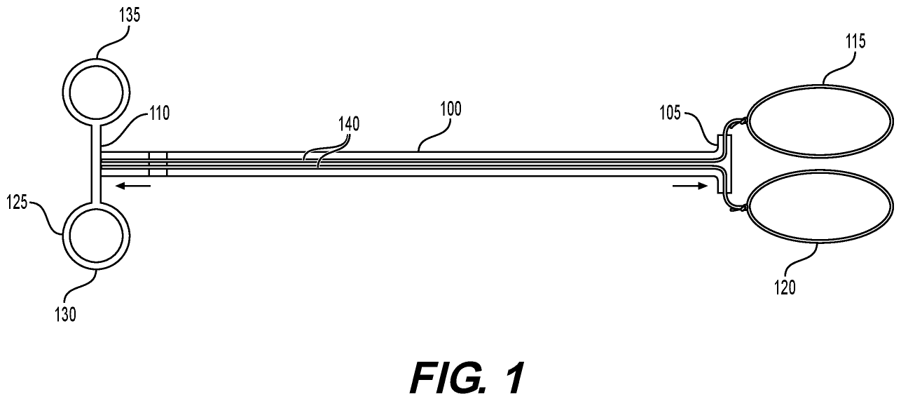

FIG. 1 is an illustration of a double endoscopic loop that includes a longitudinal body 100 having a distal end 105 and a proximal end 110. The distal end 105 includes a first loop 115 and a second loop 120. The proximal end 110 includes a handle 125 having a first looped handle 130 and a second looped handle 135. Wires 140 connect the handle 125 to first loop 115 and second loop 120.

The length of the longitudinal body 100, in some embodiments, is about 30 cm. In this regard, the longitudinal body 100 can have a length of about 25-35 cm, about 25 cm, about 26 cm, about 27 cm, about 28 cm, about 29 cm, about 30 cm, about 31 cm, about 32 cm, about 33 cm, about 34 cm, or about 35 cm. The first loop 115 can be spaced apart from the second loop 120 by a distance of about 4 cm. In this regard, the first loop 115 can be spaced apart from the second loop 120 by a distance of about 2-6 cm, about 2 cm, about 2.5 cm, about 3 cm, about 3.5 cm, about 4 cm, about 4.5 cm, about 5 cm, about 5.5 cm, or about 6 cm. The first looped handle 130 can extend about 5 cm to the right of the longitudinal body 100 and the second looped handle 135 can extend about 5 cm to the left of the longitudinal body 100. In this regard, the first and second looped handles 130, 135 can extend from the longitudinal body, in their respective directions, by about 2.5-7.5 cm, about 2.5 cm, about 3 cm, about 3.5 cm, about 4 cm, about 4.5 cm, about 5 cm, about 5.5 cm, about 6 cm, about 6.5 cm, about 7 cm, or about 7.5 cm. The wires 140 extend from the handle 125 to the first loop 115 and the second loop 120. The first loop 115 and the second loop 120 get smaller and tighten when the handle 125 is pulled away from the longitudinal body 100.

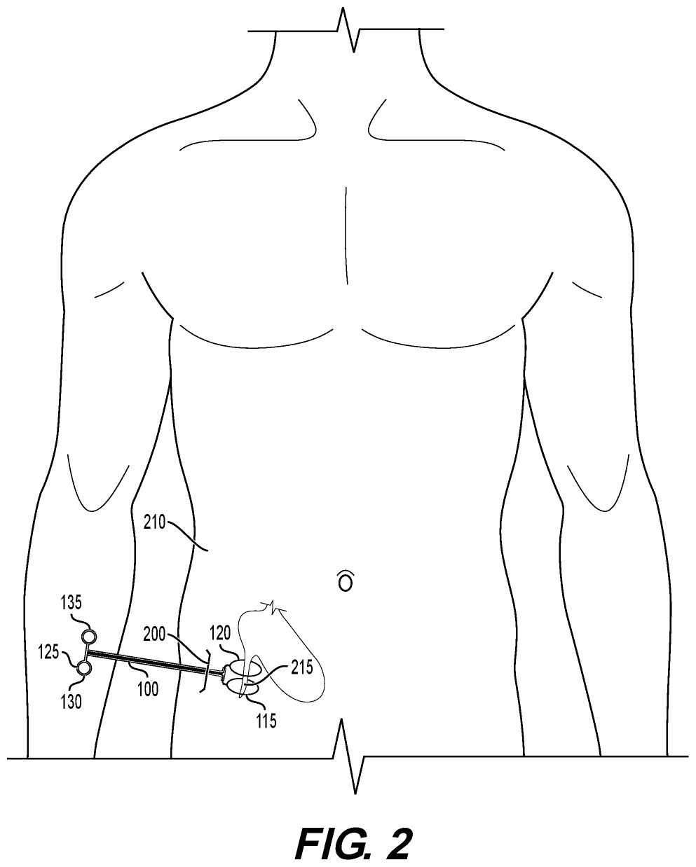

FIG. 2 is an illustration of the double endoscopic loop inside of a patient. The double endoscopic loop can be used for the ligation of the appendiceal stump during a laparoscopic appendectomy. After the dissection of the mesoappendix, the base of the appendix is prepared for ligation. An incision 200 is initially made in the body 210 of the patient. The longitudinal body 100 of the double endoscopic loop is inserted through the incision 200 into the body 210. The first loop 115 and the second loop 120 extend inside the body 210 and are placed around the appendix 215 at the appendiceal base. The handle 125 remains outside the body 210 and is accessible to the surgeon performing the appendectomy. The first looped handle 130 and the second looped handle 135 are gripped by the surgeon and pulled away from the longitudinal body 100 tightening the first loop 115 and second loop 120 to secure the appendix base. As the first loop 115 and the second loop 120 are pulled away from the longitudinal body 100, the loops get smaller and tighten. In some embodiments when one of the loops can no longer be tightened, the other loop is allowed to tighten. In other embodiments, both loops are restricted from tightening when one loop can no longer be tightened.

It is to be understood that the double endoscopic loop for laparoscopic surgery is not limited to the specific embodiments described above. For example the spacing of the loops, how the loops tighten and where cutting takes place, as disclosed, are just examples. This disclosure includes other variations, and encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A device having endoscopic loops for laparoscopic surgery, the device comprising:
a longitudinal body having a proximal end and a distal end, wherein the distal end is T-shaped and defines first and second branches, and wherein the first and second branches are axially aligned with respect to one another, each extending perpendicular to a longitudinal axis of the longitudinal body;
a first loop supported by, and extending from, the first branch of the distal end of the longitudinal body, wherein the first loop is configured for securing the appendage at a first point;
a second loop supported by, and extending from, the second branch of the distal end of the longitudinal body, the second loop being spaced apart from the first loop, wherein the second loop is configured for securing the appendage at a second point; and
a handle located at the proximal end, wherein the handle comprises a first looped handle extending between 2.5 and 7.5 cm perpendicularly from the longitudinal body in a first direction and a second looped handle extending between 2.5 and 7.5 cm perpendicularly from the longitudinal body in a second direction opposite the first direction, and wherein the handle is connected to the first loop and the second loop such that upon being pulled away from the longitudinal body the first loop ligates the appendage at the first point and the second loop ligates the appendage at the second point.

2. The device as recited in claim 1, wherein the first point is located at a base of the appendage and the second point is located at a distance on the appendage spaced apart from the first point.

3. The device as recited in claim 1, wherein the first loop is spaced apart from the second loop by a distance of between 4 and 6 cm.

4. The device as recited in claim 1, wherein the longitudinal body is between 25 and 35 cm long.

5. (canceled)

6. (canceled)

7. The device as recited in claim 1, further comprising wires, wherein the wires extend from the handle to the first loop and the second loop such that when the handle is pulled away from the longitudinal body, the first loop ligates the appendage at the first point and the second loop ligates the appendage at the second point.

8. The device as recited in claim 1, wherein the laparoscopic surgery is an appendectomy and the appendage is an appendix.

9. A method of assisting in an appendectomy in a patient, the method comprising:
making an incision in a body of the patient above an appendix of the patient;
inserting a device having endoscopic loops through the incision into the body of the patient, the device comprising:
a longitudinal body having a proximal end and a distal end, wherein the distal end is T-shaped and defines first and second branches, and wherein the first and second branches are axially aligned with respect to one another, each extending perpendicular to a longitudinal axis of the longitudinal body;
a first loop supported by, and extending from, the first branch of the distal end of the longitudinal body, wherein the first loop is configured for securing the appendage at a first point;
a second loop supported by, and extending from, the second branch of the distal end of the longitudinal body, the second loop being spaced apart from the first loop, wherein the second loop is configured for securing the appendage at a second point;
a handle located at the proximal end, the handle connected to the first loop and the second loop wherein the handle comprises a first looped handle extending between 2.5 and 7.5 cm perpendicularly from the longitudinal body in a first direction and a second looped handle extending between 2.5 and 7.5 cm perpendicularly from the longitudinal body in a second direction opposite the first direction; and
securing the appendix at a first point using the first loop and securing the appendix at a second point using the second loop; and
pulling the handle away from the longitudinal body such that the first loop ligates the appendix at the first point and the second loop ligates the appendix at the second point.

10. The method as recited in claim 9, wherein the first point is located at a base of the appendix and the second point is located at a distance on the appendix spaced apart from the first point.

11. The method as recited in claim 9, wherein the device further comprises wires that extend from the handle to the first loop and the second loop such that when the handle is pulled away from the longitudinal body, the first loop ligates the appendix at the first point and the second loop ligates the appendix at the second point.

12. The device as recited in claim 1, wherein the first and second loops lie in first and second planes, respectively, and wherein the first and second planes are parallel with respect to one another.

13. The method as recited in claim 9, wherein the first and second loops lie in first and second planes, respectively, and wherein the first and second planes are parallel with respect to one another.
